(19) United States
(12) Patent Application Publication
(10) Pub. No.: US 2018/0297726 A1
(43) Pub. Date: Oct. 18, 2018

(54) Marking of therapeutic edibles

(71) Applicant: Aari Ruben, Tucson, AZ (US)

(72) Inventor: Aari Ruben, Tucson, AZ (US)

(21) Appl. No.: 15/731,065

(22) Filed: Apr. 13, 2017

(51) Int. Cl. B65B 5/04; G09F 3/02; A61K 36/18

(52) U.S. Cl. CPC B65B 5/04; G09F 3/02; A23V 2002/00; G09F 2003/0208; A61K 36/18

(57) ABSTRACT

A packaged therapeutic edible containing a derivative of cannabis. The packaging of the therapeutic edible is adapted to minimize the potential for accidental consumption of the edible by a non-patient. Ideally, on the edible, an indicia or inscription of the therapeutic component with its dosage is imprinted onto the edible itself. Additionally, an overlay containing information on the therapeutic component is placed to encase the edible, then the entire assembly is wrapped with yet another label.

### BACKGROUND OF THE INVENTION

This invention relates generally to therapeutic edibles with particular application to the use of cannabis and its derivatives in consumables.

Within the medical industry, particularly for pills, there is little effort paid on labeling, the medicine involved nor any real indication of the strength of the medicine. Within the pill industry, a pill may be identified by the pharmacist by its shape, color, and sometimes an etched marking such as a line. This type of marking is totally useless for a “lay” person or patient. For this reason, pharmacists are frustrated when a patient attempts to describe their pill as “a little white pill”.

The sole meaningful type of identification is a label placed on the bottle or tube; but, this doesn't address the problem when a care-giver wants to ascertain if the patient's dose being applied is correct or not.

Liquids are identified within the industry solely by a label on the bottle.

Consumable products having medicinal value have been a difficult problem. Typically, these consumables are simply identified by a simple wrapping that, at best, identifies the medicine included within the consumable but does nothing to identify the dosage of that medicine.

Nowhere is this problem more pronounce than in the cannabis derivative treatment where consumables are used extensively. There have been great advancements in the identification of the medicinal benefits for derivatives as, but not limited to: Cannabidol (CBD), Cannabigerol (CBG), Cannabichromene (CBC), Cannabinol (CBN), and Tetrahydrocannabinol (THC). When these derivatives are diffused, injected, or placed in consumables such as suckers, candy, cookies, brownies, and a host of other consumables, the resulting product is often poorly identified as to the strength and content using the current techniques and can lead to being consumed by an unsuspecting consumer.

There is even a line of books and pamphlets which attempt to give guidance on how to decode the label on the package. Clearly, if the label is too confusing for a patient, imagine the confusion that would be created with an untrained individual or a child.

It is clear from the foregoing that there is a significant need for a proper and consistent marking of consumables to assure proper administration of the therapeutic drug.

### SUMMARY OF THE INVENTION

The invention is a redundant set of labeling for an edible having a therapeutic component therein. The invention is ideally suited when the edible contains a derivative of cannabis. The packaging of the therapeutic edible is adapted to minimize the potential for accidental consumption of the edible by a non-patient. Ideally, on the edible, an indicia or inscription of the therapeutic component with its dosage is imprinted/etched into the edible itself. Additionally, an overlay wrapping containing information encases the therapeutic component is placed and then the entire assembly is wrapped with yet another label.

The packaging of the edible is ideally done in an automatic fashion using any number of mechanisms. There are a variety of packing techniques and mechanisms well known to those of ordinary skill in the art, including, but not limited to that described in: U.S. Pat. No. 8,596,031, entitled “Wrap Dispensing Station and Method” issued to Pearl et al. on Dec. 3, 2013; U.S. Pat. No. 8,574,491, entitled “Unit and Method for Sterilizing a Web of Packaging Material for a Machine for Packaging Pourable Food Products” issued to Donati on Nov. 5, 2013; U.S. Pat. No. 5,908,649, entitled “Package for Perishable Food and Horticultural Products” issued to Floyd et al. on Jun. 1, 1999; U.S. Pat. No. 4,393,758, entitled “Food Processing Apparatus” issued to Anmahian on Jul. 19, 1983; and U.S. Pat. No. 4,624,100, entitled “Device for Continuously Feeding (synchronizing) Essentially Flat Articles of the Luxury-Food or Food Industry, especially Bars or Strips of Chocolate, to a Packaging Machine” issued to Lesch on Nov. 25, 1986; all of which are incorporated hereinto by reference.

It is this wrapping which assists so profoundly with the safety characteristics of the present invention. Not only does the packaging provide for a hygienic distribution mechanism, but by proper labeling on the package, the dosage and active component is clearly identified; thereby reducing the potential for accidental consumption.

In this regard, there are a variety of indices which are ideally used. In the preferred embodiment, the first indicia identifies the strength of the therapeutic component contained within the edible (e.g. “0.1 mg). This indicia is ideally pressed or etched into the edible. Imprinted on an interior wrapping (directly in contact with the edible) having a second indicia which, in the preferred embodiment corresponds the first indicia.

Surrounding the edible and the interior wrapping is an exterior wrapping which is imprinted a well to identify the active ingredient and dosage. In some embodiments, a symbol associated with cannabis is used to provide further warnings that the edible is not a “treat” but contains a derivative of cannabis requiring care in its use.

Those of ordinary skill in the art readily recognize a variety of mechanisms which can be utilized for the imprinting of labels onto the packaging, such as but not limited to: U.S. Pat. No. 5,645,300, entitled “Vegetable Label Wrapper” issued to Hill on Jul. 8, 1997; and U.S. Pat. No. 5,866,181, entitled “Label Assembly for Wrapping Vegetables” issued to Hill on Feb. 2, 1999; both of which are incorporated hereinto by reference.

Although the preferred embodiment has all three indices are substantially identical, in some embodiments the indices are not. This embodiment permits the use of specialized warnings on the interior wrap with the indicia imprinted on the consumable being only the fundamental facts about the ingredients (i.e. “CBD 0.5 mg”)

In some embodiments, an indicia includes a symbol associated with a derivative of cannabis. Those of ordinary skill in the art readily recognize a variety of symbols in this context, such as, but not limited to:

CBG Cannabigerol

CBC Cannabichromene

CBN Cannabinol

Additionally, there are a variety of terpenes which are applicable for medical application in this context.

In still other embodiments, the exterior package contains a generic label identifying the edibe as containing a derivative of cannabis.

Those of ordinary skill in the art readily recognize a variety of such cannabis related symbols such as, but not limited to: a red cross; a variety of symbols containing an outline of cannabis leaf; labels containing the words “hemp”, “cannabis”, “marijuana”, “medical marijuana”, “medical cannabis”, “THC”, “CBD”, and a host of other such labeling.

In some embodiments, where only CBD is used, the fact that the edible is devoid of any THC is noted on the label. CBD is not regulated by any governmental agency whereas THC with its affects, is.

In still other embodiments, the actual color on the exterior wrapping indicates which derivate of cannabis in infused into the edible. As example:

| | | |
|---|---|---|
| Red | THC | Tetrahydrocannabinol |
| Green | CBD | Cannabidiol |

The exterior wrapping also provides some embodiments to contain information about the proper patient for the consumable.

The invention, together with various embodiments will be more fully explained by the accompanying drawings and the following description thereof.

### DRAWINGS IN BRIEF

FIGS. 1A, 1B, and 1C illustrate the production sequence for the preferred embodiment together with an illustration of the final product.

### DRAWINGS IN DETAIL

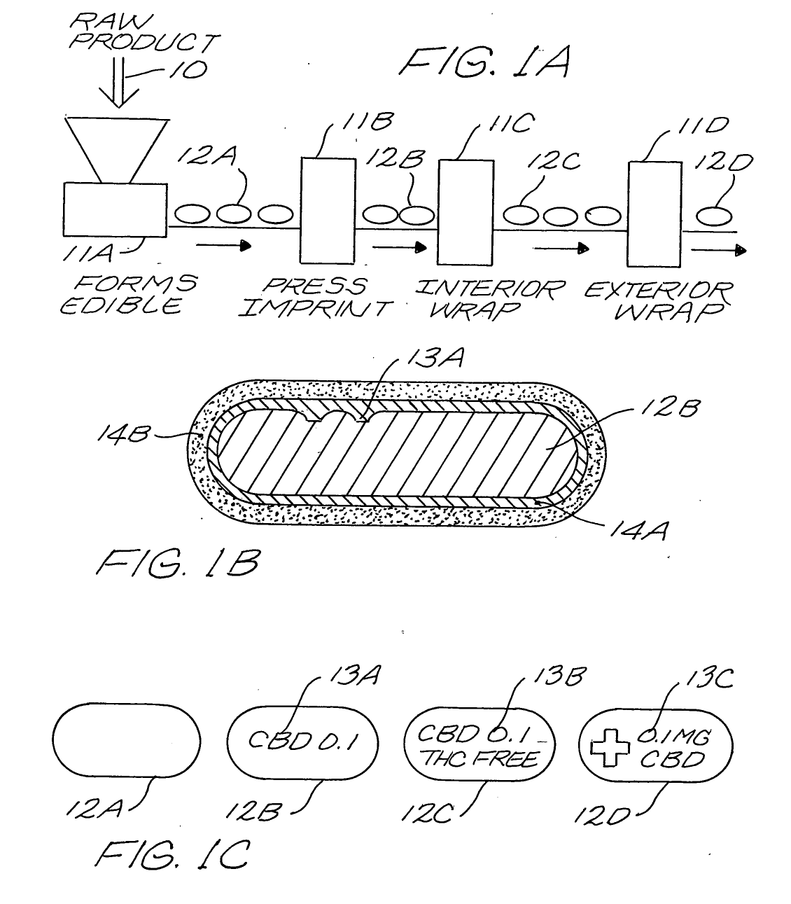

FIGS. 1A, 1B, and 1C illustrate the production sequence for the preferred embodiment together with an illustration of the final product.

Raw material 10 is deposited into the mechanism 11A which creates the edible into a preselected form 12A. Raw material 10 is typically the infused bulk edible. The form 12A taken includes a variety of shapes (a cookie in this illustration) such as a sucker, a pill, a cigarette, etc.

Mechanism 11B takes this cannabis laden product and imprints 13A (ideally through pressure although writing using an edible dye is also contemplated) to create the product 12B which is then wrapped using interior wrap 11C which applies an interior wrap 14A. The interior wrap 14A also has a printing/indicia thereon 13B.

The now partially wrapped product 12C is communicated to the exterior wrap mechanism 11D which applies an exterior wrap 14B having its own associated printing 13C to generate a final product 12D.

In this manner, the cannabis derivative product is given three level of identification to provide maximum safety.

It is clear from the foregoing that the present invention provides for proper and consistent marking of therapeutic consumables to assure proper administration of the therapeutic drug.

What is claimed is:

1. A packaged therapeutic edible comprising:
a) an edible item containing a therapeutic component infused therein, said edible item having a first indicia thereon, said first indicia identifying a strength of the therapeutic component therein;
b) a paper cover overlaying said first indicia, said paper cover having a second indicia imprinted thereon, said second indicia corresponding to the first indicia; and,
c) a wrapping encasing the edible item and the paper cover, said wrapping having a third indicia imprinted thereon, said third indicia corresponding to the first indicia and the second indicia.

2. The packaged therapeutic edible according to claim 1, wherein:
a) the first indicia is not identical to the second indicia nor the third indicia; and,
b) the second indicia is not identical to the third indicia.

3. The packaged therapeutic edible according to claim 1, wherein the wrapping contains a fourth indicia imprinted thereon, the fourth indicia being a generic label for the therapeutic component therein.

4. The packaged therapeutic edible according to claim 3, wherein:
a) the therapeutic component is a derivative of cannabis; and,
b) the fourth indicia includes a symbol associated with a derivative of cannabis.

5. The packaged therapeutic edible according to claim 4, wherein the derivative of cannabis is devoid of tetrahydrocannabinol.

6. The packaged therapeutic edible according to claim 4, wherein the third indicia includes a coloring of the wrapping.

7. The packaged therapeutic edible according to claim 6, wherein the color of the wrapping is indicative of the derivative of cannabis infused within the edible item.

8. The packaged therapeutic edible according to claim 7, wherein the coloring of the wrapping is substantially consistent throughout the exterior of the wrapping.

9. The packaged therapeutic edible according to claim 1, wherein the first indicia and the second indicia are substantially identical.

10. An edible comprising:
a) an edible item infused with a derivative of cannabis, said edible item having a first indicia therein, said first indicia identifying a strength of the derivative of cannabis therein; and,
b) An exterior wrapping encasing the edible item, said wrapping having a second indicia imprinted thereon, said second indicia on the exterior wrapping corresponding to the first indicia.

11. The packaged therapeutic edible according to claim 10, wherein the derivative of cannabis is devoid of tetrahydrocannabinol.

12. The edible according to claim 10, further including a paper cover having an indicia corresponding to the first indicia on the edible item, said paper cover interposed between the edible item and the exterior wrapping.

13. The edible according to claim 12, wherein the exterior wrapping has a colored exterior surface to indicate a strength of the derivative of cannabis.

14. A packaged therapeutic edible comprising:
a) an edible item having,
1) a therapeutic component infused therein, and,
2) an imprinted labeling thereon;
b) a cover encasing the edible item and having information imprinted thereon concerning the therapeutic component; and,
c) a wrapping encasing the edible item and the paper cover and having information concerning the therapeutic component including a dosage thereof.

15. The packaged therapeutic edible according to claim 14, wherein,
a) the therapeutic component is a derivative of cannabis; and,
b) the wrapping includes a graphic symbol associated with the derivative of cannabis.

16. The packaged therapeutic edible according to claim 15, wherein the wrapping has a colored exterior surface being chosen to indicated the dosage of the derivative of cannabis.

17. The packaged therapeutic edible according to claim 16, wherein the information on the wrapping is indicative of a patient for the therapeutic edible.

18. The packaged therapeutic edible according to claim 17, wherein the derivative of cannabis is devoid of tetrahydrocannabinol.
